(19) United States
(12) Patent Application Publication
(10) Pub. No.: US 2023/0371917 A1
(43) Pub. Date: Nov. 23, 2023

(54) AUSCULTATION WEARABLE WITH MECHANICAL AMPLIFIER AND OFFSET ACOUSTIC TRANSDUCERS

(71) Applicant: The George Washington University, Washington, DC (US)

(72) Inventors: R. Scott DOWNEN, Arlington, VA (US); Zhenyu LI, McLean, VA (US)

(21) Appl. No.: 18/030,449

(22) PCT Filed: Oct. 6, 2021
(86) PCT No.: PCT/US2021/053789

§ 371 Date: Apr. 5, 2023

Related U.S. Application Data
(60) Provisional application No. 63/088,163, filed on Oct. 6, 2020.

(51) Int. Cl. A61B 7/04; A61B 5/00

(52) U.S. Cl. CPC A61B 7/04; A61B 5/7203; A61B 5/7475; A61B 2562/166; A61B 2562/0204

(57) ABSTRACT

A wearable includes: a mechanical amplifier; a first acoustic transducer positioned to sense acoustic signals from a target surface and amplified by the mechanical amplifier, the first acoustic transducer configured to generate a first set of electrical signals based on the sensed acoustic signals; a second acoustic transducer offset from the first acoustic transducer and positioned to sense ambient acoustic noise, the second acoustic transducer configured to generate a second set of electrical signals; a microcontroller coupled to the first and second acoustic transducers; and a transmitter coupled to the microcontroller. The microcontroller is configured to prepare a data set based on a digitized version of the first and second sets of electrical signals. The transmitter is configured to: receive the data set from the microcontroller; and transmit the data set to another device.

### CROSS-REFERENCE TO RELATED APPLICATIONS

This application claims priority to U.S. Provisional Application No. 63/088,163, filed Oct. 6, 2020, which is hereby incorporated by reference.

### BACKGROUND

Acoustic sensors are used in many applications. One example application of acoustic sensors is auscultation. Traditional auscultation techniques rely on stethoscopes. More recently, digital stethoscopes capable of recording and transmitting auscultation audio have been commercialized. There have also been efforts to leverage available smartphone components (e.g., microphone, processor, storage) for use with auscultation. To date, auscultation wearables have not been commercialized. Some of the challenges related to commercializing auscultation wearables include signal-to-noise ratio (SNR) limitations (e.g., interference from ambient noises), size limitations, and power supply limitations.

### SUMMARY

In one example embodiment, a wearable includes: a mechanical amplifier; a first acoustic transducer positioned to sense acoustic signals from a target surface and amplified by the mechanical amplifier, the first acoustic transducer configured to generate a first set of electrical signals based on the sensed acoustic signals; a second acoustic transducer offset from the first acoustic transducer and positioned to sense ambient acoustic noise, the second acoustic transducer configured to generate a second set of electrical signals; a microcontroller coupled to the first and second acoustic transducers; and a transmitter coupled to the microcontroller. The microcontroller is configured to prepare a data set based on a digitized version of the first and second sets of electrical signals. The transmitter is configured to: receive the data set from the microcontroller; and transmit the data set to another device.

### BRIEF DESCRIPTION OF THE DRAWINGS

FIG. 1 is a block diagram of an auscultation wearable in accordance with an example embodiment.

FIG. 2 is an exploded-view showing an auscultation wearable in accordance with an example embodiment.

FIG. 3 is a cross-sectional view showing a mechanical amplifier for an auscultation wearable in accordance with another example embodiment.

FIG. 4 is a cross-sectional view of an auscultation wearable in accordance with an example embodiment.

FIG. 5 is an top-view showing circuitry of an auscultation wearable in accordance with an example embodiment.

FIGS. 6A-6C are graphs showing synchronous data including auscultation data obtained by an auscultation wearable in accordance with an example embodiment.

FIG. 7 is a diagram showing features of an auscultation wearable in accordance with an example embodiment.

FIG. 8 is a flowchart showing an auscultation wearable method in accordance with an example embodiment.

The same reference numbers (or other reference designators) are used in the drawings to designate the same or similar (structurally and/or functionally) features.

### DETAILED DESCRIPTION

Some example embodiments include an auscultation wearable or patch having: offset acoustic transducers; a mechanical amplifier configured to amplify frequencies of interest; and electronics configured to: store one or more time intervals (e.g., 5-15 seconds) of electrical signals generated by the acoustic transducers; and perform other operations (e.g., electronic amplification or attenuation of certain frequencies, audio pattern analysis, preparation of a data set, transmission of the data set to another device, etc.). As used herein, a “wearable” or “patch” refers to a module or unit that attaches to a target surface and may be flat, curved, flexible, or otherwise optimized in shape to maximize contact with the target surface. The particular shape and size of the wearable or patch may vary. Example dimensions for a wearable or patch using width (W), length (L), and height (H) are: W of 1.5″ or less; L of 1.5″ or less; and H of 0.5″ or less. Hereafter, the term “wearable” rather than “wearable or patch” is used throughout the description. While the auscultation wearable options described herein are suitable for auscultation scenarios, it should be appreciated that the same or similar device could be used in other acoustic sensing applications such as monitoring or recording the acoustics of a pipe (indicating fluid flow, passage of air bubbles, cavitation, blockages, or other features of interest).

Without limitation, an auscultation wearable may include various features. For example, each acoustic transducer of an auscultation wearable may be a microelectromechanical system (MEMS) piezoelectric microphone. Also, the mechanical amplifier may have a bell shape or truncated cone shape that forms a chamber, where at least one of the acoustic transducers either resides in the chamber or is near the chamber to sense acoustic signals amplified by the mechanical amplifier. To filter or otherwise tune the acoustic signals captured by the auscultation wearable, the wall thickness and/or surface features of the mechanical amplifier may vary. In some example embodiments, the mechanical amplifier includes a surface with stepped or ribbed configuration features (e.g., ribs that extend around an interior or external surface of the mechanical amplifier), where the stepped or ribbed configuration features result in the amplification or attenuation of certain frequencies. Other frequency tuning and filtering options include: use of a filler in the chamber; use of a membrane between the mechanical amplifier and the target surface to which the auscultation wearable is attached; and/or use of frequency damping materials on the interior and/or exterior of the mechanical amplifier. As another option, the auscultation wearable includes a housing make from a sound-blocking material and/or a sound-blocking material may be placed on the exterior surface of the housing to reduce ambient noise.

Without limitation, the operations of an auscultation wearable is combinable with the operations of other sensors (e.g., a pulse monitor, an electrocardiogram (ECG) source, or other sensors) and their respective data sets can be synchronized together and provided as a combined data set. Such combined data sets can improve detection of a health alert or otherwise improve diagnosis accuracy. As another option, multiple auscultation wearables may be used together and the respective data sets combined to improve the diagnosis accuracy. In different example embodiments, the amount of processing and/or audio pattern detection performed by an auscultation wearable may vary. One example auscultation wearable may be configured to capture and transfer full audio to another computing device for later analysis or consideration by the other computing device or its operators (e.g., medical personnel). Another example auscultation wearable may be configured to capture certain frequencies and perform audio pattern detection on the captured frequencies to identify one or more alerts (e.g., an alert is generated in response to a predetermined audio pattern and/or a threshold). Any alerts and/or a related audio pattern identifier may be transferred by the auscultation wearable to a local or remote computing device via a wired or wireless communication channel for consideration by the other computing device or its operators. In response to an alert, medical personnel may respond by performing follow-up auscultation using a traditional stethoscope and/or other diagnosis operations are performed. Other auscultation wearable options include: a user interface configured to enable start/stop options, provide relevant indicators (e.g., battery levels, alerts, etc.), or other options; use of differential noise cancellation (sometimes referred to as active-noise cancellation) techniques based on the electrical signals obtained by offset acoustic transducers of an auscultation wearable to improve the signal-to-noise ratio (SNR) of frequencies of interest; and electrical frequency tuning or filtering options (to amplify and/or attenuate certain frequencies). The electrical tuning or filtering options may be programmable in some embodiments. Various other options are possible as described in relation to the figures herein.

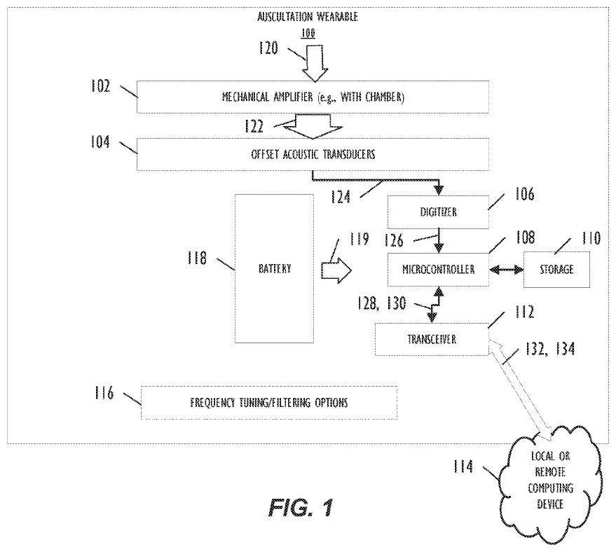

FIG. 1 is a block diagram of an auscultation wearable 100 in accordance with an example embodiment. The auscultation wearable 100 may take the form of a modular unit that can be adhered to a patient, a pipe, or another source of acoustic signals. As shown, the auscultation wearable 100 includes a mechanical amplifier 102 and offset acoustic transducers 104. In operation, the mechanical amplifier 102 is configured to amplify at least certain frequencies of ambient acoustic signals 120, resulting in amplified acoustic signals 122 that are sensed by the offset acoustic transducers 104. When the auscultation wearable 100 is attached to a target surface (e.g., a patient's chest or back, a pipe, or other target surface), the ambient acoustic signals 120 includes target audio signals as well as ambient noise.

In some example embodiments, the mechanical amplifier 102 has a bell or truncated conical shape that tapers outward from a narrower proximal end to a wider distal end. A chamber is formed between the proximal end and distal end of the mechanical amplifier 102, and the offset acoustic transducers 104 may be within or near the chamber formed by the mechanical amplifier 102. With the offset acoustic transducers 104, exposure of each acoustic transducer to target audio signals and ambient noise varies, which enables differential noise cancellation operations. In response to sensing acoustic signals including the amplified acoustic signals 122, the offset acoustic transducers 104 generate electrical signals 124. The electrical signals 124 include signal amplitudes and frequency components, which include the amplified acoustic signals 122 subject to some imperfection inherent in the offset acoustic transducers 104. The electrical signals 124 are provided to a digitizer 106, resulting in digitized audio signals 126 based on the electrical signals 124. As shown, other components of the auscultation wearable 100 include: a microcontroller 108 coupled to the digitizer 108; storage 110 (e.g., random-access memory, flash memory, or other memory) coupled to or included with the microcontroller 108; a transceiver 112 coupled to the microcontroller 108; and a battery 118 configured to provide power 119 to active circuitry of the auscultation wearable 100. In some example embodiments, the battery 118 is rechargeable. In such case, the auscultation wearable 100 may additionally include battery charge control circuitry.

In the example of FIG. 1, the digitized audio signals 126 generated by the digitizer 106 are provided to the microcontroller 108. In some example embodiments, the microcontroller 108 is configured to store a number of time intervals (e.g., 5 to 15 seconds) of digitized audio signals 126 in the storage 110. As another option, the microcontroller 108 may analyze the digitized audio signals 126 to identify audio patterns of interest. In such embodiments, discrete Fourier transform (DFT) operations and/or other analysis options may be used to perform acoustic spectroscopy operations on the digitized audio signals 126.

In response to a request, trigger, or schedule, the time intervals of digitized audio signals 126 and/or acoustic spectroscopy results (e.g., data and/or alerts) are provided as a data set 128 to the transceiver 112. The transceiver 112 provides the data set 128 to a local or remote computing device 114 as data packets 132 via a wired or wireless communication protocol. Example wireless protocols that may be used by the transceiver include Bluetooth Low Energy (BLE) or cellular wireless protocols.

In some example embodiments, the microcontroller 108 unidirectionally communicates with a local or remote computing device 114 (e.g., a smartphone or other handheld device, a laptop, a desktop computer, a server, etc.) either directly or via a network. In such case, the transceiver 112 could be replaced by a transmitter. In other example embodiments, the microcontroller 108 bidirectionally communicates, via the transceiver 112, with the local or remote computing device 114 either directly or via a network. Example networks include a wireless local area network (WLAN), a cellular network, or a global network such as the Internet. Without limitation, the microcontroller 108 may: send data to the local or remote computing device 114; receive data from the local or remote computing device 114; or receive control signals from the local or remote computing device 114. In some scenarios, a first local or remote computing device initiates monitoring operations of the auscultation wearable 100, and a second local or remote computing device receives or analyzes monitoring results from the auscultation wearable 100.

In some example embodiments, the transceiver 112 is configured to receive data packets 134 from the local or remote computing device 114. The data packets 134 may include instructions for the microcontroller 108. Example instructions include an auscultation type identifier (e.g., heart, arteries, or lungs), frequencies of interest, control signals (e.g., a time interval duration, a start command, a stop command, transfer and/or delete stored audio recordings, transfer and/or delete stored analysis results, etc.) and/or other information. Such information in the data packets 134 may be recovered by the transceiver 112 and provided to the microcontroller 108 as a data set 130. With the ability to receive information from the local or remote computing device 114 or a related network, the microcontroller 108 and related auscultation operations are programmable.

In different example embodiments, the amount of processing performed by the microcontroller 108 varies. In some example embodiments, the digitized audio signals 126 obtained by the auscultation wearable 100 are analyzed by the local or remote computing device 114. In such embodiments, the auscultation wearable 100 may forego differential noise cancellation operations and analysis of the digitized audio signals 126 and related circuitry. In such embodiments, the local or remote computing device 114 may perform these operations based on the digitized audio signals 126. In other example embodiments, the auscultation wearable 100 performs differential noise cancellation operations, but does not further analyze the digitized audio signals 126. In still other example embodiments, the auscultation wearable 100 performs differential noise cancellation operations and analysis of the digitized audio signals 126. In such case, the local or remote computing device 114 does not perform analysis of the digitized audio signals 126, but may forward related audio recordings or analysis results, store related audio recordings or analysis results, play back related audio recordings, and/or display the related analysis results.

In the example of FIG. 1, the auscultation wearable 100 includes frequency tuning/filtering options 116. The frequency tuning/filtering options 116 may be mechanical and/or electrical. Without limitation, the frequency tuning/filtering options 116 may include: selection of a material, size, shape, and/or surface features for the mechanical amplifier 102 to target a predetermined frequency response; selection of a material, size, shape, and/or surface features for a membrane (coupling the auscultation wearable 100 to a target surface such as a patient's skin or the surface of a pipe) to target a predetermined frequency response; variations in the amount or type of filler in the chamber formed by the mechanical amplifier 102 and membrane to target a predetermined frequency response; selection of coatings/fillers for the interior and/or exterior of the mechanical amplifier 102 to target a predetermined frequency response; selection of a material, size, shape, and/or surface features for a housing for the auscultation wearable 100 to target a predetermined frequency response; and/or selection of coatings/fillers for the interior and/or exterior of the housing to target a predetermined frequency response. Other options for the auscultation wearable 100 include: varying the number of and the position of the offset acoustic transducers 104; use of different noise cancellation techniques to improve SNR of acoustic signals of interest relative to ambient noise; and user interface options (e.g., an on/off button, a start/stop button, indicator lights, etc.).

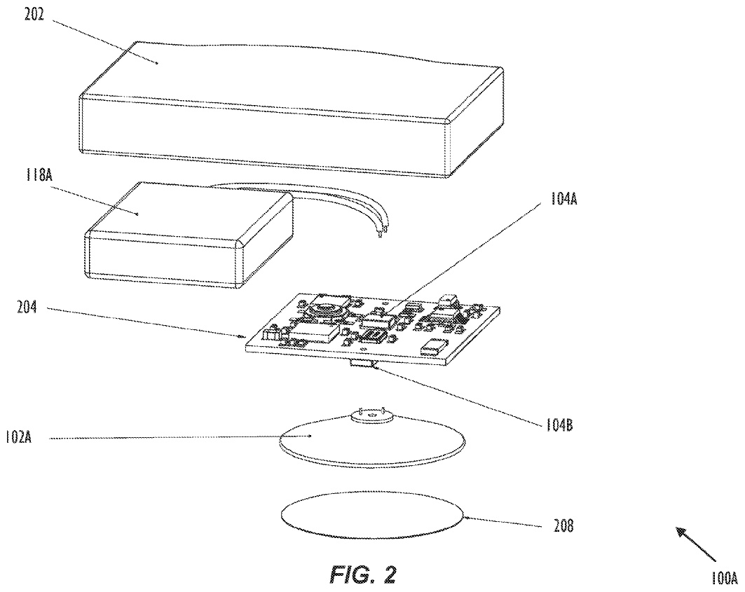

FIG. 2 is an exploded-view showing an auscultation wearable 100A (an example of the auscultation wearable 100 in FIG. 1) in accordance with an example embodiment. In FIG. 2, various components of the auscultation wearable 100A are displayed including a housing 202, a battery 118A (an example of the battery 118 in FIG. 1), a printed circuit board (PCB) assembly 204, a first acoustic transducer 104A (e.g., one of the offset acoustic transducers 104 in FIG. 1), a second acoustic transducer 104B (e.g., another of the offset acoustic transducers 104 in FIG. 1), a mechanical amplifier 102A (an example of the mechanical amplifier 102 in FIG. 1), and a membrane 208. When the displayed components of the auscultation wearable 100A are assembled, the housing 202 and the membrane 208 are visible while the other components are internal to the housing 202 and membrane 208. In some example embodiments, the membrane 208 is arranged within an opening at the base of the housing 202 such that the membrane 208 is at (e.g., protruding, flush or recessed with respect to) the outer surface of the housing 202.

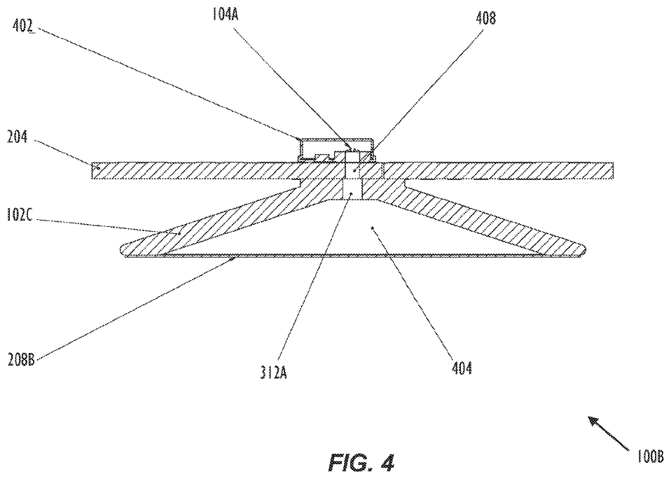

Without limitation to other example embodiments, the housing 202 may be a flexible silicone housing, the battery 118A may be a 250 mAh capacity rechargeable Lithium-Ion Polymer (LiPo) battery cell, and the mechanical amplifier 102A may be an aluminum bell or truncated cone configured to provide mechanical amplification of sound waves. Further, the first acoustic transducer 104A may be a MEMS piezoelectric microphone within or near the chamber of the mechanical amplifier 102A. In operation, the first acoustic transducer 104A converts ambient sound waves interacting with the membrane 210 and amplified through the mechanical amplifier 102A into voltages, which are read by a microcontroller (e.g., the microcontroller 108 in FIG. 1) included with the PCB assembly 204. Without limitation to other example embodiments, the second acoustic transducer 104B may be a MEMS piezoelectric microphone offset from the first acoustic transducer 104A. The second acoustic transducer 104B also converts ambient sound waves into voltages, which are read by a microcontroller (e.g., the microcontroller 108 in FIG. 1) included with the PCB assembly 204. The voltages provided by the first acoustic transducer 104A and the second acoustic transducer 104B enable differential noise reduction (sometimes called active-noise cancellation) operations by the microcontroller 108A. In some example embodiments, the membrane 208 is configured to couple the auscultation wearable 100A to a target surface (e.g., a patient's skin) and helps create an enclosed chamber (see e.g., chamber 404 in FIG. 4). Without limitation, the membrane 208 may be an epoxy/fiberglass blend.

In some example embodiments, the interior of the housing 202 is partly or completely filled with a filler such as noise isolating foam. As another option, filler may surround the PCB assembly 204 within the housing 202 to provide mechanical dampening of noise created by bodily movement. Other filler options include: use of foam between the mechanical amplifier 102A and the PCB assembly 204; and use of a spray or hydrogel-based foam to fill any voids in the housing 202.

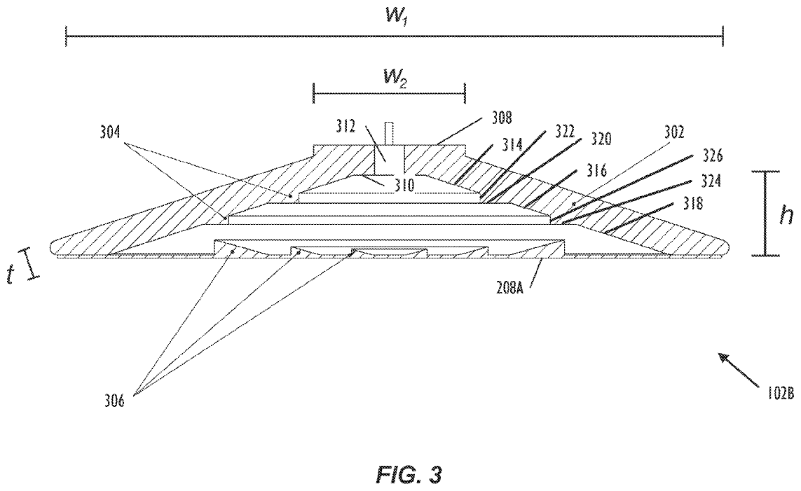

FIG. 3 is a cross-sectional view showing a mechanical amplifier 102B (an example of the mechanical amplifier 102 in FIG. 1) and a membrane 208A (an example of the membrane 208 in FIG. 2) for an auscultation wearable (e.g., the auscultation wearable 100 in FIG. 1, or the auscultation wearable 100A in FIG. 2) in accordance with another example embodiment. In the example of FIG. 3, the mechanical amplifier 102B has a truncated cone shape and is defined by a number of parameters including the ratios of W₁/W₂ and W₂/h. Here W₁ is a first diameter related to a bottom (i.e., wide distal end) of the truncated cone shape, W₂ a second diameter related to a top (i.e., narrow proximal end) of the truncated cone shape, and h is the height of the interior of the truncated cone shape. The height is parallel to a longitudinal axis of the mechanical amplifier 102B, and the diameters are perpendicular to the longitudinal axis. The truncated cone shape of the mechanical amplifier 102B also includes a wall 302 with a thickness (t). In different example embodiments, W₁, W₂, h, and t may vary to target the acoustic frequencies that are amplified or attenuated by the mechanical amplifier 102B. Without limitation, an example of the mechanical amplifier 102B may have W₂=0.20″, W₁=0.874″, h=0.149″, and t=0.02″. In some example embodiments, W₁, W₂, h, and/or t are selected to account for known frequency responses present in human hearing to create a more hearable frequency response. Also, the shape and/or size of the mechanical amplifier 102B may vary in different example embodiments, which would result in changes to the amplified and/or attenuated frequencies. As desired, the shape and/or size of the mechanical amplifier 102B may be selected for use with a particular auscultation scenario (e.g., obtaining tuned audio related to hearts, arteries, or lungs).

In some example embodiments, the mechanical amplifier 102B includes surface features and/or wall thickness features to amplify and/or attenuate target frequencies. In some example embodiments, the surface features and/or wall thickness features result in stepped or ribbed configuration features 304 (e.g., ribs along the interior surface of the mechanical amplifier 102B) to provide mechanical damping and filtration of select sound frequencies. The stepped or ribbed configuration features 304 are either an integral part of the wall 302 of the mechanical amplifier 102B or are mechanically coupled to the wall 302 of the mechanical amplifier 102B. In the example of FIG. 3, each of the stepped or ribbed configuration features 304 extends around the interior surface of the mechanical amplifier 102B. For example, the stepped or ribbed configuration features 304 may form concentric rings or circles along the interior surface of the mechanical amplifier 102B. Such features may be optimized in simulation to provide filtration and amplification at desired frequencies to target particular auscultation applications (e.g., heart sounds, specific heart sounds, lung sounds, specific lung sounds, arterial blood flow, etc.). In some example embodiments, the mechanical amplifier 102B is made from aluminum or an aluminum alloy. In some example embodiments, the material used for the mechanical amplifier 102B may be selected based on simulation of sound wave propagation through the mechanical amplifier 102B.

More specifically, as shown in FIG. 3, the wall 302 of the mechanical amplifier 102B includes a base 308 at the proximal end of the mechanical amplifier 102B. The base 308 includes a forward-facing inner surface 310 and a central through-hole 312. In some example embodiments, the wall 302 is a single integral material with a linear inner surface that tapers outward from the proximal end of the mechanical amplifier 102B to the distal end of the mechanical amplifier 102B. The stepped or ribbed configuration features 304 are formed as tabs or ribs that project inward from the inner surface of the wall 302, and have a leading surface and a trailing surface. The leading surface of the stepped or ribbed configuration features 304 extend directly inward in a transverse direction perpendicular to the longitudinal axis of the mechanical amplifier 102B, each leading surface forming a shelf portion. The trailing surface of the stepped or ribbed configuration features 304 extend in a longitudinal direction at a right angle to the leading surface, parallel to the longitudinal axis of the mechanical amplifier 102B, each trailing surface forming a lip portion.

In the example of FIG. 3, the stepped or ribbed configuration features 304 define a plurality of sections of the mechanical amplifier 102B including: a first proximal section 314 positioned about the central through-hole 312; a second intermediate section 316 positioned at an intermediate portion of the mechanical amplifier 102B; and a third distal section 318 positioned about the distal end of the mechanical amplifier 102B. The first proximal section 314 has a proximal end that is larger/wider than the central through-hole 312, which is located at the proximal end of the mechanical amplifier 102B. Thus, the proximal end of the first proximal section 314 forms the forward inner-facing surface 310. The distal end of the first proximal section 314 forms a first shelf that extends in a first transverse direction (substantially horizontal in the example embodiment of FIG. 3). More specifically, the sides of the first proximal section 314 are angled or tapered outward and include a tapered portion 320 that is tapered to form the first shelf, and a vertical portion 322 that extends in a second direction (i.e., substantially vertical in the example embodiment of FIG. 3) that is substantially orthogonal to the first transverse direction. Similarly, the second intermediate section 316 has a proximal end that is larger/wider than the distal end of the first proximal section 314 to form the first shelf. In addition, the distal end of the second intermediate section 316 forms a second shelf that extends in the first transverse direction (substantially horizontal in the example embodiment of FIG. 3). The sides of the second intermediate section 316 are angled or tapered outward and include a tapered portion 324 that is tapered to form the second shelf, and a vertical portion 326 that extends in the second direction (i.e., substantially vertical in the example embodiment of FIG. 3) substantially orthogonal to the first transverse direction.

In the example of FIG. 3, the membrane 208A also includes stepped or ribbed configuration features 306 to provide mechanical damping and filtration of select sound frequencies. The stepped or ribbed configuration features 306 are either an integral part of the membrane 208A or are mechanically coupled to the membrane 208A. In some example embodiments, each of the stepped or ribbed configuration features 306 forms a circle shape on one side of the membrane 208A (e.g., the stepped or ribbed configuration features 306 form a set of concentric circles or ribs on one side of the membrane 208A). Such features of the membrane 208A may be optimized in simulation to provide filtration and amplification at desired frequencies to target particular auscultation applications (e.g., heart sounds, specific heart sounds, lung sounds, specific lung sounds, arterial blood flow, etc.).

FIG. 4 is a cross-sectional view showing an auscultation wearable 100B (an example of the auscultation wearable 100 in FIG. 1, or components of the auscultation wearable 100A in FIG. 2) in accordance with another example embodiment. In the cross-sectional view of FIG. 4, the auscultation wearable 100B includes: the first acoustic transducer 104A; the PCB assembly 204; a mechanical amplifier 102C (an example of the mechanical amplifier 102 in FIG. 1, or the mechanical amplifier 102A in FIG. 2); and a membrane 208B (an example of the membrane 208 in FIG. 2). In the example of FIG. 4, the mechanical amplifier 102C has a featureless or smooth interior surface. In other example embodiments, the auscultation wearable 100B may include a mechanical amplifier and/or membrane with stepped or ribbed configuration features (see e.g., the mechanical amplifier 102B and membrane 208A in FIG. 3).

In the example of FIG. 4, there is a chamber 404 between an interior surface of the mechanical amplifier 102C and the membrane 208B. In different example embodiments, the chamber 404 is empty or is partly or completely filled with a material based on its acoustic frequency response. Example fillers includes: argon, spray foam, hydrogel, or similar materials to create a mechanical low-pass filter. As shown, the mechanical amplifier 102C includes a through-hole 312A (an example of the central through-hole 312 in FIG. 3), which forms an audio port. Also, the PCB assembly 204A includes a through-hole that forms an audio port 408. As shown, the through-hole 312A is aligned with the audio port 408. As another option, the first acoustic transducer 104A may be within an enclosure 402. In different example embodiments, the size of the through-hole 312A and the audio ports 406 and 408 may vary. Such variance may be based on target diameters or lengths related to Helmholtz frequencies or design criteria to provide mechanical filtration of sound waves.

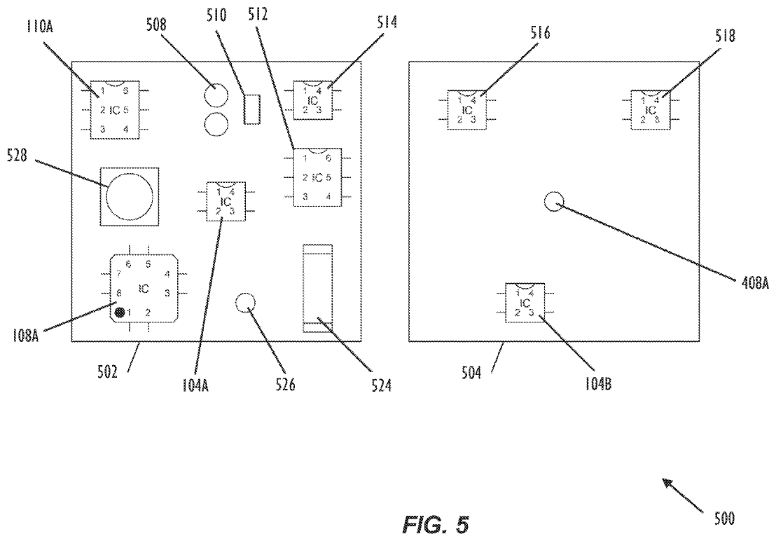

FIG. 5 is a top-view showing circuitry 500 of an auscultation wearable (e.g., the auscultation wearable 100 in FIG. 1, the auscultation wearable 100A in FIG. 2, or the auscultation wearable 100B in FIG. 4) in accordance with an example embodiment. In some example embodiments, the circuitry 500 includes PCBs 502 and 504, which may correspond to the PCB assembly 204 in FIG. 2, or the PCB assembly 204A in FIG. 4. In other example embodiments, a single PCB may be used. As shown, the components or features of the PCBs 502 and 504 include: flash memory 110A (an example of the storage 110 in FIG. 1); a battery connection interface 508; a tri-color Light-Emitting Diode (LED) 510; an operational amplifier 512 (e.g., used in a filtration and amplification stage related to the first and second acoustic transducers 104A and 104B); and a low-dropout regulator (LDO) 514 used to provide a stable power supply (e.g., 3V) to PCB components from a battery (not shown). The components or features of the PCBs 502 and 504 further include: a dual field-effect transistor (FET) shutoff circuit 516 used along with a battery monitor integrated circuit (IC) 518 for circuit protection; and an audio port 408A (an example of the audio port 408 in FIG. 4). When a related auscultation wearable is assembled, the audio port 408A is aligned with the through-hole (e.g., the through-hole 312 in FIG. 3, or the through-hole 312A in FIG. 4) of a mechanical amplifier as described herein.

In some example embodiments, the components or features of the PCBs 502 and 504 additionally include: the first acoustic transducer 104A and the second acoustic transducer 104B, which are offset from each other. In some example embodiments, the offset between the first acoustic transducer 104A and the second acoustic transducer 104B is in a transverse direction relative a longitudinal axis of a related auscultation wearable. In other example embodiments, the offset between the first acoustic transducer 104A and the second acoustic transducer 104B is in an axial direction relative a longitudinal axis of a related auscultation wearable. In still other example embodiments, the offset between the first acoustic transducer 104A and the second acoustic transducer 104B includes transverse direction and axial direction offsets relative a longitudinal axis of a related auscultation wearable.

With the first acoustic transducer 104A and the second acoustic transducer 104B offset from each other, ambient noise is better distinguished from target audio for the purpose of active-noise cancellation. The components or features of the PCBs 502 and 504 additionally include: an antenna 524, an audio port 526 used to pass ambient acoustic signals to the second acoustic transducer 104B; a microcontroller 108A (an example of the microcontroller 108 in FIG. 1); and a micro-tactile switch 528 used for user interaction and on-demand audio sampling. In some example embodiments, the PCB 502 is positioned above the PCB 504. In other example embodiments, the positioning of PCB components or the position of the PCBs relative to each other may vary. It is also possible to use a single PCB with components on one side or both sides of the PCB.

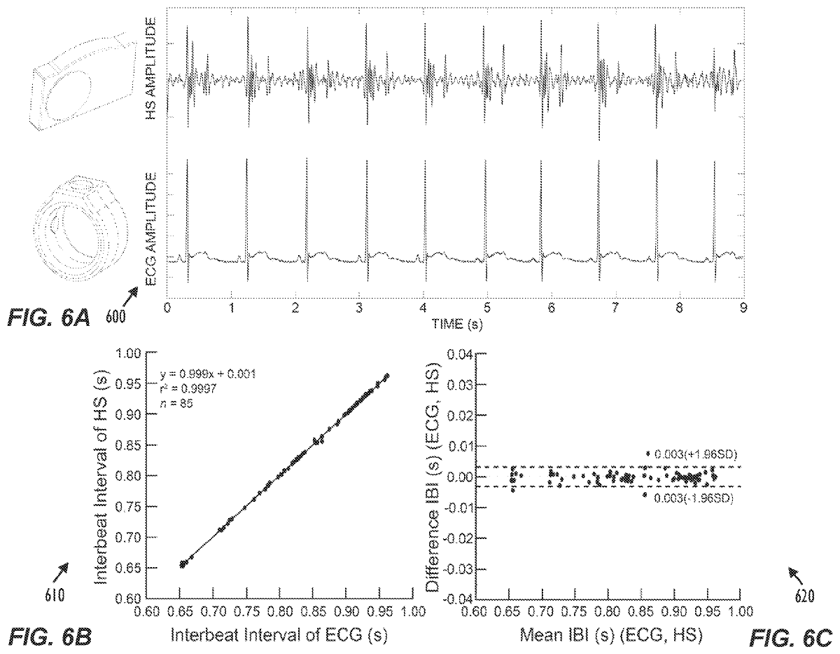

FIGS. 6A-6C are graphs 600, 610, and 620 showing auscultation parameters in accordance with an example embodiment. In graph 600 of FIG. 6A, sample heart-sound (HS) data obtained by an auscultation wearable (e.g., the auscultation wearable 100 in FIG. 1, the auscultation wearable 100A in FIG. 2, or the auscultation wearable 100B in FIG. 4) is displayed synchronously with an electrocardiogram (ECG) obtained by another sensor (e.g., a finger worn ring). One such reconfigurable flexible device is shown, for example, in U.S. Pat. No. 11,013,462, the entire contents of which are incorporated herein by reference. The synchronous HS data and ECG data can be indicative of cardiopulmonary conditions. In graph 610 of FIG. 6B, correlative data from 85 heartbeats is displayed showing strong correlation between heart-sound peak-to-peak intervals and electrical peak-to-peak intervals. In graph 620 of FIG. 6C, a sample Bland-Altman plot is displayed demonstrating analysis which can be completed based on comparing heart sounds to electrical activity. In some example embodiments, data obtained by an auscultation wearable is combined with data obtained by one or more other sensors to facilitate identifying health issues of interest.

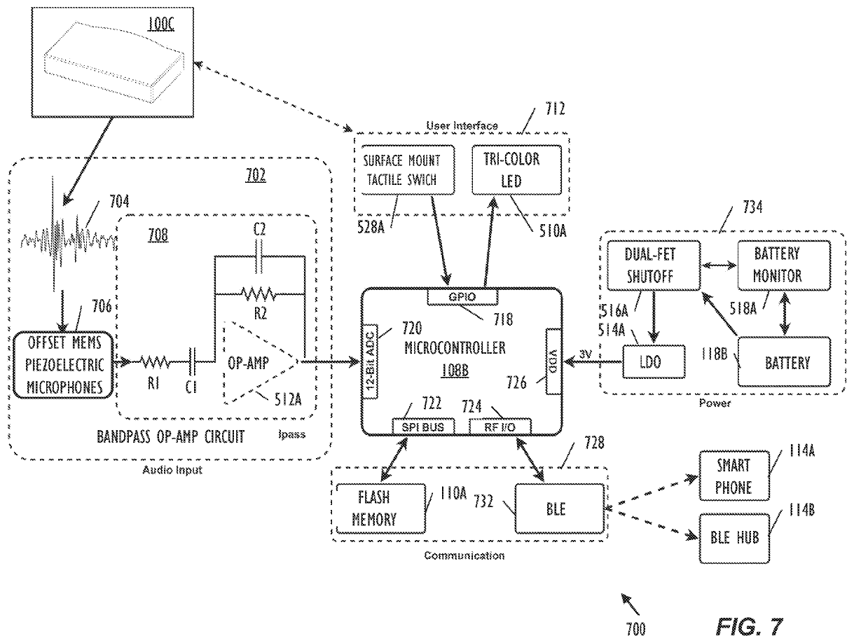

FIG. 7 is a diagram 700 showing features of an auscultation wearable 1000 (an example of the auscultation wearable 100 in FIG. 1, the auscultation wearable 100A in FIG. 2, or a housed version of the auscultation wearable 100B in FIG. 4) in accordance with an example embodiment. In the diagram 700, the features are given as: audio input 702; a microcontroller 108B (an example of the microcontroller 108 in FIG. 1, or the microcontroller 108A in FIG. 5); a user interface 712; a communication interface 728; and a power interface 734.

In the diagram 700, the auscultation wearable 1000 obtains acoustic data 704 using offset MEMS piezoelectric microphones 706 (examples of the offset acoustic transducers 104 in FIG. 1, the first acoustic transducer 104A in FIGS. 2, 4, and 5, or the second acoustic transducer 104B in FIGS. 2 and 5). The acoustic data 704 is filtered by a bandpass op-amp circuit 708 that includes an operational amplifier 512A (an example of the operational amplifier 512 in FIG. 5), resistors (R1 and R2), and capacitors (C1 and C2) in the arrangement shown. In different example embodiments, the same or different bandpass op-amp circuit as the bandpass op-amp circuit 708 is used for each of the offset MEMS piezoelectric microphones 706. The audio input 702 (the output of the operational amplifier 710 or each such operational amplifier) is provided to an analog-to-digital converter (ADC) interface 720 of the microcontroller 108B. In this example, the microcontroller 108B includes its own digitizer (e.g., the digitizer 106 in FIG. 1).

As shown, the user interface 712 includes a surface-mount tactile switch 528A (an example of the surface-mount tactile switch 528 in FIG. 5) and a tri-color LED 510A (an example of the tri-color LED 510 in FIG. 5) coupled to a general-purpose input/output (GPIO) interface 718 of the microcontroller 108B. In some example embodiments, the tri-color LED 510A provides feedback options for the end user (e.g., indicating active audio recording, data transfer, fault conditions, or system status indications such as low-battery alerts or charge complete). In some example embodiments, the surface-mount micro-tactile switch 528A is used to initiate on-demand audio samples.

The power interface 734 includes a battery 118B (an example of the battery 118 in FIG. 1, or the battery 118A in FIG. 2), a battery monitor IC 518A (an example of the battery monitor IC 518 in FIG. 5), a dual-FET shutoff circuit 516A (an example of the dual-FET shutoff circuit 516 in FIG. 5), and an LDO 514A (an example of the LDO 514 in FIG. 5). With the power interface 734, the LDO 514A is configured to provide power to a power supply (VDD) interface 726 of the microcontroller 108B. The condition of the battery 118B is monitored using the battery monitor IC 518A. As needed, the dual-FET shutoff circuit 516A may shutoff (effectively removing the battery 118B from the downstream circuit) in response to a trigger (e.g., an overcurrent condition, overcharge condition, or a discharge condition) identified the battery monitor IC 518A. In some example embodiments, the auscultation wearable 1000 is powered by a rechargeable LiPo battery. In some example embodiments, the LDO 514A provides a stable 3V supply to active components of the auscultation wearable 1000 from the battery 118B.

In the diagram 700, the communication interface 728 includes the flash memory 110A and a BLE module 732. The flash memory 110A is coupled to a serial peripheral interface (SPI) bus 722 of the microcontroller 108B. The BLE module 732 is coupled to a radio-frequency input/output (RF I/O) of the microcontroller 724. After obtaining digitized audio signals, the microcontroller 108B is configured to store and/or analyze the digitized audio signals. The stored digitized audio signals and/or analysis results may be transferred via the BLE module 728 to a smart phone 114A and/or a BLE hub 114B (examples of the local or remote computing device 114 in FIG. 1). In other example embodiments, a wired connection to a local computing device is possible. In still other example embodiments, a cellular or other long-range wireless connection to a remote computing device is possible. As another option, collected audio data or analysis results is buffered through the microcontroller 108B into the flash memory 110A for later recall. In some use scenarios, data may be pulled from flash memory 110A immediately for transfer to backend devices (e.g., the smartphone 114A, the BLE hub 114B, a laptop, a desktop computer, or other networked devices) for further analysis.

In some example embodiments, the microcontroller 108B is used for all data collection, data transfer, and user interactions. In the diagram 700, audio collected from by the auscultation wearable 1000 is converted from acoustic-energy to electrical-energy in the form of voltages by the offset MEMS piezoelectric microphones 706. The electrical signals are then filtered and amplified using the operational amplifier 512A. As desired, amplification and filtration values can be altered by changing the values of R1, R2, C1, and C2 depending on the specific use case. Use of variable resistors and varactors is possible for R1, R2, C1, and C2. For heart sounds, expected amplification and frequencies are lower (e.g., gain below 150× and a frequency range around 20 Hz-1 kHz). For lung sounds, expected amplification and frequency ranges are higher (e.g., gain above 20× and a frequency range around 20 Hz-4 kHz). In some example embodiments, the auscultation wearable 1000 is tuned for heart sounds, with a gain of around 122× and a bandpass frequency range of around 19 Hz to 2.34 kHz. Another option is to collect a large frequency range with a high sampling frequency and use software filtering on the back-end as needed. In this use case, a physician may be able to select “heart” or “lung” sounds, for example, and the software automatically filters the collected audio for these desired signals. Similarly, digitally adjustable hardware of the auscultation wearable 1000 may be tuned via software. As another option, a digital-potentiometer can be used to alter electronic filtration options of the auscultation wearable 1000.

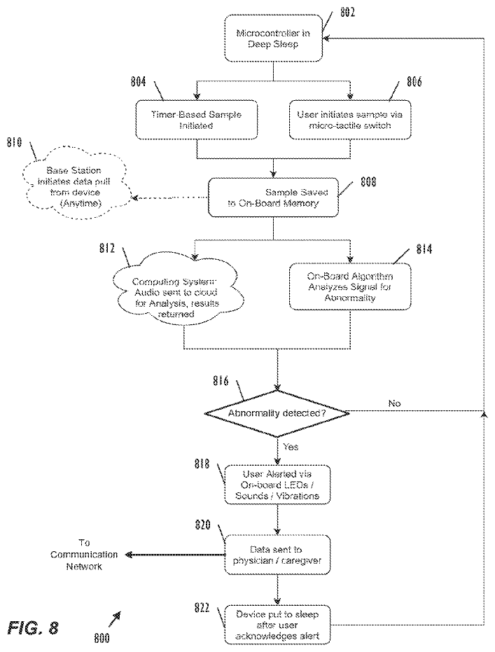

FIG. 8 is a flowchart showing an auscultation wearable method 800 in accordance with an example embodiment. The method 800 begins at block 802 with the microcontroller (e.g., the microcontroller 108 in FIG. 1, the microcontroller 108A in FIG. 5, or the microcontroller 108B in FIG. 7) in a deep sleep state. At block 804, a timer-based sample is initiated. As another option, a user initiates a sample (e.g., using the micro-tactile switch) at block 806. In either case, a sample corresponding to a time interval (e.g., 5 to 15 seconds) is saved to on-board memory at block 808. At block 810, a base station, smartphone, or other computing device is able to initiate a data pull from the auscultation wearable at any time. At block 812, audio is sent to a computing system (e.g., cloud computing) for analysis. In some example embodiments, the results are returned to the auscultation wearable. As another option, an on-board algorithm of the auscultation wearable analyzes the saved sample for abnormalities at block 814. In either case, if an abnormality is not detected (decision block 816), the method 800 returns to block 802. If an abnormality is detected (decision block 816), a user may be alerted via on-board LEDs, audible signals, and/or vibrations at block 818. At block 820, data is sent (e.g., via email, text, or other alert mechanism) to a physician or caregiver via a communication network. At block 822, the auscultation wearable is put to sleep after the user acknowledges the alert and the method 800 returns to block 802. In some example embodiments, the user may silence an alarm by pressing the on-board micro-tactile switch, which puts the auscultation wearable back into a deep sleep. As needed, older data may be deleted from the memory of the auscultation wearable to make room for new samples.

While the mechanical amplifier is noted to be conical in certain embodiments, other suitable shapes can be used. In addition, while the described wearable is utilized for medical purposes, other suitable applications are possible (e.g., to listen to fluid flow in pipes).

In this description, the term “couple” may cover physical or electrical connections, communications, or signal paths that enable a functional relationship consistent with this description. For example, if device A generates a signal to control device B to perform an action: (a) in a first example, device A is coupled to device B by direct connection; or (b) in a second example, device A is coupled to device B through intervening component C if intervening component C does not alter the functional relationship between device A and device B, such that device B is controlled by device A via the control signal generated by device A.

A device that is “configured to” perform a task or function may be configured (e.g., programmed and/or hardwired) at a time of manufacturing by a manufacturer to perform the function and/or may be configurable (or re-configurable) by a user after manufacturing to perform the function and/or other additional or alternative functions. The configuring may be through firmware and/or software programming of the device, through a construction and/or layout of hardware components and interconnections of the device, or a combination thereof.

A circuit or device that is described herein as including certain components may instead be adapted to be coupled to those components to form the described circuitry or device. For example, a structure described as including one or more semiconductor elements (such as transistors), one or more passive elements (such as resistors, capacitors, and/or inductors), and/or one or more sources (such as voltage and/or current sources) may instead include only the semiconductor elements within a single physical device (e.g., a semiconductor die and/or integrated circuit (IC) package) and may be adapted to be coupled to at least some of the passive elements and/or the sources to form the described structure either at a time of manufacture or after a time of manufacture, for example, by an end-user and/or a third-party.

Circuits described herein are reconfigurable to include the replaced components to provide functionality at least partially similar to functionality available prior to the component replacement. Components shown as resistors, unless otherwise stated, are generally representative of any one or more elements coupled in series and/or parallel to provide an amount of impedance represented by the shown resistor. For example, a resistor or capacitor shown and described herein as a single component may instead be multiple resistors or capacitors, respectively, coupled in parallel between the same nodes. For example, a resistor or capacitor shown and described herein as a single component may instead be multiple resistors or capacitors, respectively, coupled in series between the same two nodes as the single resistor or capacitor. Unless otherwise stated, “about,” “approximately,” or “substantially” preceding a value means +/−10 percent of the stated value.

It is noted that the drawings may illustrate, and the description and claims may use geometric or relational terms, such as longitudinal axis, transverse, side, top, bottom, linear, parallel, perpendicular, circular, conical, etc. These terms are not intended to limit the disclosure and, in general, are used for convenience to facilitate the description based on the examples shown in the figures. In addition, the geometric or relational terms may not be exact. For instance, walls may not be exactly perpendicular or parallel to one another because of, for example, roughness of surfaces, tolerances allowed in manufacturing, etc., but may still be considered to be perpendicular or parallel.

Modifications are possible in the described embodiments, and other embodiments are possible, within the scope of the claims. For example, although the auscultation wearables described herein use wireless technology to communicate with a network, wired interfaces for data transfer and/or power transfer are possible

What is claimed is:

1. A wearable, comprising:
a mechanical amplifier;
a first acoustic transducer positioned to sense acoustic signals from a target surface and amplified by the mechanical amplifier, the first acoustic transducer configured to generate a first set of electrical signals based on the sensed acoustic signals;
a second acoustic transducer offset from the first acoustic transducer and positioned to sense ambient acoustic noise, the second acoustic transducer configured to generate a second set of electrical signals;
a microcontroller coupled to the first and second acoustic transducers and configured to prepare a data set based on a digitized version of the first and second sets of electrical signals; and
a transmitter coupled to the microcontroller and configured to:
receive the data set from the microcontroller; and
transmit the data set to another device.

2. The wearable of claim 1, wherein the transmitter is part of a wireless transceiver configured to transmit the data set to the other device via a wireless communication channel.

3. The wearable of claim 1, wherein the data set includes a time interval of auscultation audio obtained from a patient.

4. The wearable of claim 1, wherein the data set includes a time interval of audio signals obtained from a pipe.

5. The wearable of claim 1, wherein the data set includes an alert based on audio pattern recognition.

6. The wearable of claim 1, wherein the data set includes acoustic data synchronized with other sensor data.

7. The wearable of claim 1, wherein the mechanical amplifier has a surface with stepped or ribbed configuration features.

8. The wearable of claim 1, wherein the mechanical amplifier is made from aluminum or an aluminum alloy.

9. The wearable of claim 1, wherein the mechanical amplifier has a bell shape or truncated cone shape with a proximal end and a distal end.

10. The wearable of claim 9, wherein the mechanical amplifier includes an audio port at the proximal end of the bell shape or truncated cone shape.

11. The wearable of claim 10, wherein the audio port is a first audio port, the wearable device further comprises a printed circuit board (PCB) assembly that includes a first PCB and a second PCB, the first PCB having a second audio port aligned with the first acoustic transducer, and the second PCB having a third audio port aligned with the second acoustic transducer.

12. The wearable of claim 1, further comprising:
a battery;
a voltage regulator between the battery and the microcontroller; and
a digitizer coupled to the first acoustic transducer, the second acoustic transducer and the microcontroller, wherein the digitized is configured to:
receive the first set of electrical signals from the first acoustic transducer;
receive the second set of electrical signals from the second acoustic transducer; and
provide the digitized version of the first and second sets of electrical signals to the microcontroller.

13. The wearable of claim 1, wherein the microcontroller is configured to perform active-noise cancellation based on the digitized version of the first and second sets of electrical signals.

14. The wearable of claim 1, further comprising a membrane positioned along a base of the mechanical amplifier, wherein the mechanical amplifier and the membrane form a chamber.

15. The wearable of claim 14, wherein the membrane has stepped or ribbed configuration features.

16. The wearable of claim 14, further comprising a filler material within the chamber, wherein the filler material is selected based on its acoustic frequency response.

17. The wearable of claim 1, further comprising a user interface coupled to the microcontroller, the user interface having a button and indicators.

18. The wearable of claim 1, further comprising a frequency tuning circuit configured to amplify or attenuate frequencies of the first and second sets of electrical signals.

19. The wearable of claim 1, wherein the first acoustic transducer and the second acoustic transducer are microelectromechanical system (MEMS) piezoelectric microphones.

20. The wearable of claim 1, further comprising a flexible silicon housing that encloses the mechanical amplifier, the first acoustic transducer, the second acoustic transducer, and the microcontroller.
